(19) United States
(12) Patent Application Publication
(10) Pub. No.: US 2021/0251480 A1
(43) Pub. Date: Aug. 19, 2021

(54) DEVICES, SYSTEMS, AND METHODS FOR AN INSTRUMENT ACCESSORY

(71) Applicant: Boston Scientific Scimed, Inc., Maple Grove, MN (US)

(72) Inventors: Javier Palomar-Moreno, Galway (IE); Darshan Dugad, Galway (IE); John Kilcooley, Gort (IE)

(21) Appl. No.: 17/177,394

(22) Filed: Feb. 17, 2021

Related U.S. Application Data
(60) Provisional application No. 62/978,018, filed on Feb. 18, 2020.

(51) Int. Cl. A61B 1/31; A61B 1/00; A61B 1/018

(52) U.S. Cl. CPC A61B 1/31; A61B 1/018; A61B 1/00082

(57) ABSTRACT

The present disclosure, in its various aspects, is directed to instrument accessory devices, implementation methods, and related delivery systems. Embodiments according to the present disclosure, including as described herein, may increase the effectiveness and efficiency of colonoscopy procedures, such as the removal of polyps. In one example, an embodiment includes an instrument accessory device with an expandable member, the device configured to receive an instrument through an instrument lumen, wherein the inner surface of the expandable member comprises a material characterized by a first elasticity and the outer surface of the expandable member comprises a material characterized by a second elasticity, the second elasticity being more elastic than the first elasticity.

### PRIORITY

The present application is a non-provisional of and claims the benefit of priority under 35 USC § 119 to U.S. Provisional Application Ser. No. 62/978,018, filed Feb. 18, 2020, the disclosure of which is herein incorporated by reference in its entirety.

### FIELD

The present disclosure pertains to medical devices. More particularly, the present disclosure pertains to instrument accessory devices and related systems and methods, particularly as may increase the effectiveness and efficiency of colonoscopy procedures, such as the removal of polyps

### BACKGROUND

Endoscopes are long, flexible instruments used in medical procedures to examine and treat conditions within the body. An example of an endoscope is a colonoscope, used for colonoscopies. Due to the length of the colonoscope and the anatomy of the colon, during some procedures, for example during polyp removal, the colonoscope can become deformed, or “loop” within the colon, causing pain for the patient and prolonging the procedure time. When looping occurs, the potential movements of the endoscopist may become limited. Consequently, it may become necessary to restart the colonoscopy procedure and the effectiveness and efficiency of the procedure may be compromised. Although endoscopists are trained in methods to reduce the prevalence of looping, it remains a recurring issue.

It is with the above considerations in mind that the improvements of the present disclosure may be useful.

### SUMMARY

The present disclosure, in its various aspects, is directed generally to instrument accessory devices, implementation methods, and related systems. Embodiments according to the present disclosure, including those described herein, may increase particularly the effectiveness and efficiency of procedures used for the examination and treatment of conditions within the body, e.g., removal of polyps within the colon through a colonoscope.

In an aspect, a device may include an expandable member having a proximal end, a distal end, and an instrument lumen extending longitudinally therethrough, the expandable member comprising an inner surface defining the instrument lumen and an outer surface disposed about the inner surface. The inner surface may comprise a material characterized by a first elasticity and the outer surface comprises a material characterized by a second elasticity, the second elasticity being greater than the first elasticity.

In various embodiments described here or otherwise, the instrument lumen may be configured to receive the instrument when the expandable member is in an expanded configuration. The instrument lumen may be configured to maintain frictional contact with the instrument when the expandable member is in a collapsed configuration. The instrument lumen may be configured to allow translational movement of the instrument when the expandable member is in the expanded configuration. When the expandable member may comprise the collapsed configuration, an expandable member diameter, an instrument lumen diameter, or both correspond to a diameter of the instrument, and, when the expandable member comprises the expanded configuration, the expandable member diameter, the instrument lumen diameter, or both, are larger the diameter of the instrument. The inner surface and outer surface may be joined to form a closed chamber. The device may further comprise a fluid inlet. A length of the expandable member along a longitudinal axis may be coextensive with a length of the instrument along the longitudinal axis. The instrument may comprise a colonoscope.

In an aspect, a system may include a colonoscope. The system may include a colonoscope accessory disposable about the colonoscope. The colonoscope accessory may comprise an expandable member having a proximal end, a distal end, and an instrument lumen extending longitudinally therethrough, the expandable member comprising an inner surface defining the instrument lumen and an outer surface disposed about the inner surface. The inner surface may comprise a material characterized by a first elasticity and the outer surface comprises a material characterized by a second elasticity, the second elasticity being more elastic than the first elasticity. The instrument lumen may be configured to slidingly receive at least a portion of the colonoscope when the expandable member is in an expanded condition.

In various embodiments described here or otherwise, the expandable member may maintain frictional contact with the colonoscope when the expandable member is in a collapsed condition. The inner surface may comprise a material characterized by a first elasticity and the outer surface comprises a material characterized by a second elasticity, the second elasticity being more elastic than the first elasticity. The material of the outer surface may be compliant and configured to expand to conform in all or part to a body cavity. When the expandable member is in the expanded condition, an inner diameter of the instrument lumen may allow the colonoscope to move freely within the instrument lumen. The expandable member may be pneumatically, electrically, or mechanically expandable.

In an aspect, a method of performing a procedure within a body lumen may comprise advancing an instrument at least partway into the body lumen. The instrument may comprise an instrument accessory device having an expandable member disposed about at least a distal portion of the instrument, the expandable member having a proximal end, a distal end, and an instrument lumen extending longitudinally therethrough. The expandable member may comprise an inner surface defining the instrument lumen and an outer surface disposed about the inner surface. The expandable member may have a collapsed configuration wherein one or both of an expandable member diameter and an instrument lumen diameter may correspond to a diameter of the instrument and an expanded configuration wherein one or both of the expandable member diameter and the instrument lumen diameter are larger than the diameter of the instrument. The method may comprise expanding the expandable member to the expanded configuration, and continuing to advance or retract the instrument through the instrument lumen within the body lumen while the expandable member is in the expanded configuration.

In various embodiments described here or otherwise, expanding the expandable member within the body lumen may expand an inner diameter of the instrument lumen such that the instrument is able to move within the instrument lumen of the expandable member. Expanding the expandable member may allow the instrument to translate within the instrument lumen. The step of expanding the expandable member may comprise injecting a gas or a liquid into the expandable member. The step of collapsing the expandable member may comprise removing a gas or liquid from the expandable member. The outer surface may comprise a compliant material that may be expandable to conform to the body lumen. Expansion of an outer diameter of the expandable member may inhibit movement of the expandable member within the body lumen. The body lumen may comprise a colon and the instrument may comprise a colonoscope. The proximal end of the expandable member may remain outside of the body lumen during the procedure. Expanding the expandable member may preferentially expand an outer diameter of the expandable member.

In an aspect, the outer diameter may expand. Expanding the outer diameter may not expand the inner diameter. The instrument may remain in frictional contact with the inner surface of the expandable member.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1A illustrates an exemplary colonoscope of a type described in an embodiment of the present disclosure.

FIG. 1B illustrated an exemplary colonoscope system of a type described in an embodiment of the present disclosure.

FIG. 2A illustrates a partial cross-sectional view of a colonoscope system with an instrument accessory device in a collapsed configuration within a body and an exemplary colonoscope, such as the colonoscope of FIG. 1, extended therethrough, in accordance with an embodiment of the present disclosure.

FIG. 2B illustrates a partial cross-sectional view of a colonoscope system with an instrument accessory device, such as the device of FIG. 2A, in an expanded configuration within a body and an exemplary colonoscope, such as the colonoscope of FIG. 1, extended therethrough, in accordance with an embodiment of the present disclosure.

FIG. 3A illustrates a perspective view of an instrument accessory device in a collapsed configuration, in accordance with an embodiment of the present disclosure.

FIG. 3B illustrates a cross-sectional view of the instrument accessory device of FIG. 3A, in a collapsed configuration.

FIG. 4A illustrates a perspective view of an instrument accessory device in a fully expanded configuration, in accordance with an embodiment of the present disclosure.

FIG. 4B illustrates a cross-sectional view of the instrument accessory device of FIG. 4A in an expanded configuration.

FIG. 5 illustrates a perspective view of a colonoscope system in a collapsed configuration, in accordance with an embodiment of the present disclosure.

FIG. 6 illustrates a perspective view of a colonoscope system during insertion of the colonoscope into an instrument accessory device, in accordance with an embodiment of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Various embodiments according to the present disclosure are described below. As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The detailed description should be read with reference to the drawings, which are not necessarily to scale, depict illustrative embodiments and are not intended to limit the scope of the invention.

The present disclosure discloses various embodiments of a device including an instrument accessory for use with an instrument such as an endoscope or the like. The instrument accessory may be included as part of an endoscope system comprising one or more of an endoscope, a handle, the instrument accessory, and an inflation fluid, or the like. In various embodiments, the instrument accessory may comprise an expandable member having an instrument lumen extending longitudinally therethrough. The instrument accessory may have an expanded configuration wherein an outer surface of the expandable member conforms to and/or dilates a body lumen, where the term conform refers to the outer surface expanding to be fully flush against the body lumen, or sufficiently so as to inhibit movement. The expandable member may be inflated to the expanded configuration during an medical procedure, such as a colonoscopy or the like. It is appreciated that the ability to dilate the colon during the colonoscopy procedure may enable the operator to uncoil loops in the colon and/or colonoscope. In some embodiments, in the expanded configuration, a diameter of the instrument lumen may be concomitantly enlarged, enabling free translation of the colonoscope through the instrument assembly, further enabling loop correction. Translational movement may occur once the expandable member is sufficiently inflated. The expandable member should be inflated only to the point where translational movement of the instrument is allowed. In some embodiments, the expandable member may be expanded using an inflation fluid comprising a gas or a liquid. In some embodiments, the body lumen may include a lumen, organ, vessel, passage, or the like, within, e.g., the digestive system, or the like.

Endoscopies, such as colonoscopies are common medical procedures used to examine the digestive system for issues such as rectal or intestinal bleeding, abdominal pain, cancer screenings, and polyps. A colonoscope is introduced into the patient via the rectum and advanced through the large intestine. The colonoscope may be used to take samples of tissue as well as visually examining the area. While moving through the large intestine, the colonoscope may form loops (e.g. ‘N’ or spiral, alpha, and deep transverse loops), which prevent movement of the colonoscope and cause pain for the patient, as well as prolonging the procedure.

For various uses of colonoscopes or other instruments, within various body lumens and for various purposes, such as described above, embodiments of the accessory devices, systems and methods of the present disclosure may be utilized to enable the physician to perform procedures with greater precision, accuracy, and ease than without the accessory devices.

In one embodiment, an instrument accessory device may include an expandable member having an instrument lumen extending therethrough. The expandable member may include an inner surface defining the instrument lumen which may be comprised of a semi-compliant material such as a balloon, a mesh, a braid, a flap, or the like. The expandable member may include an outer surface which may be a compliant elastic material such as a balloon, a mesh, a braid, a flap, or the like. The inner surface and outer surface of the expandable member may make up a balloon. In various embodiments, the expandable member may be mechanically actuated, electrically actuated, pneumatically actuated, inflated, for the transition from a collapsed configuration to an expanded configuration. In the expanded configuration, the expandable member may occlude, stretch, establish patency, or maintain patency of a body lumen.

The instrument accessory device may have a fluid inlet that may extend through the outer surface of an expandable member into a chamber defined by the inner and outer surfaces. The fluid inlet may accept an inflation fluid for expanding and un-expanding or collapsing the expandable member.

According to one method of performing a colonoscopy, a distal end of a colonoscope may be advanced through the large intestine via the rectum of a patient. Once inside the patient, the digestive system may be visually examined and tissue may be removed for biopsy or testing using an instrument passed through a working channel from a handle at the proximal end of the colonoscope that remains outside of the body.

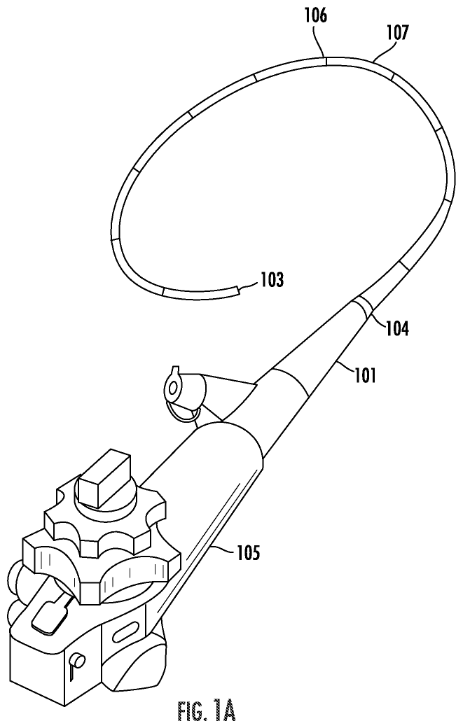

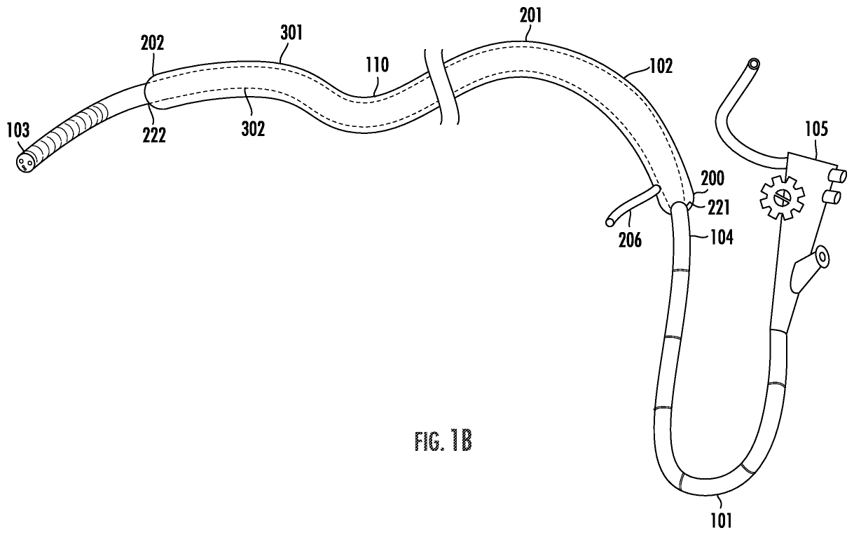

FIG. 1A illustrates one embodiment of a colonoscope which may be used with an instrument assembly as disclosed herein. The colonoscope 101 comprises a distal end 103 and a proximal end 104 with a lumen or working channel extending therethrough. A handle 105 at the proximal end 104 may be operated by a medical professional to manipulate the colonoscope 101. The colonoscope 101 may include cuts or channels 106 along a wall 107 of the insertion portion (e.g., flexible tubing) of the colonoscope 101 in order to facilitate movement and flexibility of the colonoscope 101 within a patient, e.g., by operation of steering knobs at the handle 105.

FIG. 1B illustrates one embodiment of a colonoscope system 110 according to an embodiment of the present disclosure, including an instrument accessory device 102 slidably disposed around a colonoscope 101. The instrument accessory device 102 includes an expandable member 201 and a fluid inlet 206. The expandable member 201 is shown disposed about the colonoscope 101, extending from a proximal end 104 to a distal end 103 of the colonoscope 101. The colonoscope extends into the inlet 221 of the expandable member and out of the outlet 222. The inner surface 302 lies flush with the colonoscope 101, with the outer surface 301 surrounding the inner surface 302. In an embodiment, the colonoscope 101 may comprise a handle 105 to steer the length of the colonoscope 101 through a patient. The expandable member 102 extends a majority of the length of the insertion member of colonoscope 101 distal to the handle, from the proximal end 200 to the distal end 202. The length of the colonoscope 101 may be between 1600 mm and 2100 mm, with an outer diameter of between 11.5 mm and 15 mm. The length of the instrument accessory device 102 may be similar to the colonoscope 101.

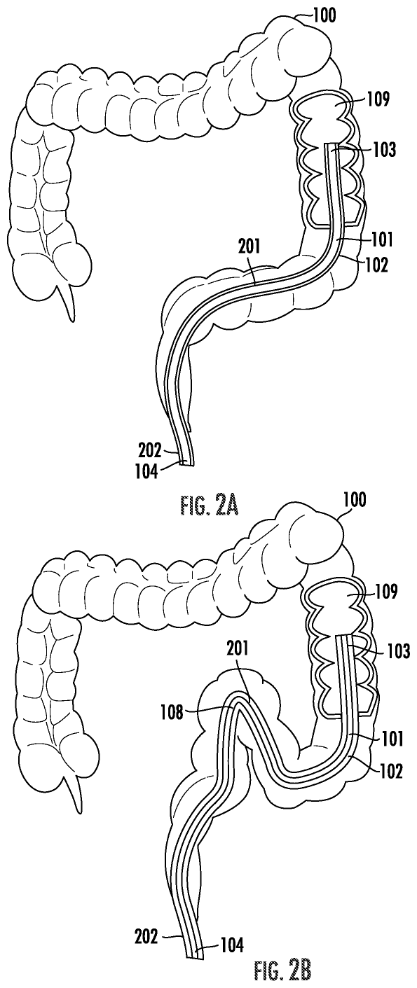

FIGS. 2A and 2B illustrate a view of the colonoscope system 110 according to an embodiment of the present disclosure within the intestine 100 wherein the colonoscope system 110 includes an instrument accessory device 102 slidably disposed around a colonoscope 101. The distal end 103 of the colonoscope system 110 may be advanced through the intestine 100, with the proximal end 104 of the colonoscope system 110 remaining outside the body. As long as no loops are formed in the colonoscope 101, the instrument accessory device 102 may remain disposed in its collapsed or deflated configuration about the colonoscope 101, where the instrument lumen and/or inner surface of the expandable member 201 generally lie flush against the colonoscope 101, each having a diameter that generally corresponds to the external diameter of the colono scope. In the event that a loop 108 forms in the colonoscope 101, the instrument accessory device 102 may be inflated (FIG. 2B). The expandable member 201 of the device 102 may substantially dilate a body lumen 109 while in the inflated configuration. The expandable member 201 may be transitioned from a collapsed configuration to an inflated configuration by supply of an inflation fluid through a fluid inlet extending through the outer surface of the expandable member 201. The body lumen 109, e.g., large intestine, may be substantially occluded by the expandable member 201 when the expandable member 201 is in an expanded state. When the expandable member 201 is in the expanded state, the diameter of the instrument lumen further expands to a diameter larger than the external diameter of the colonoscope. As a result, expansion enables the colonoscope 101 to freely translate within the instrument lumen.

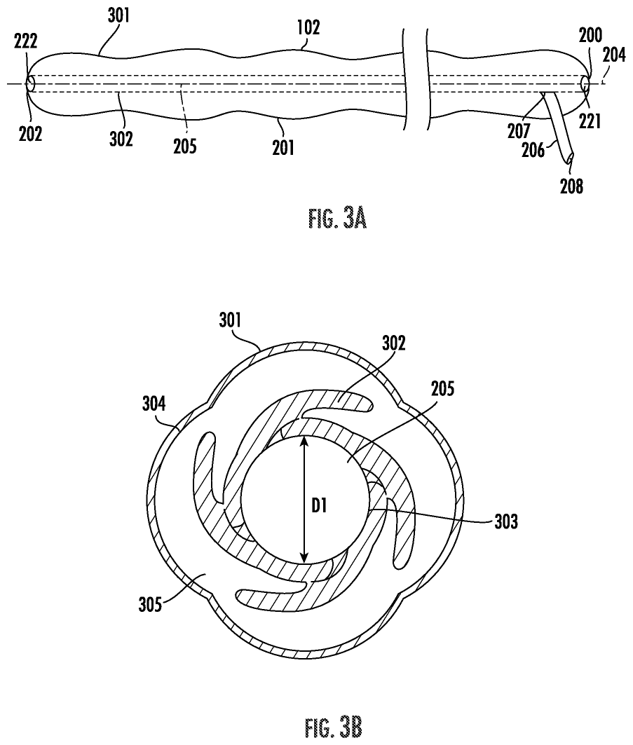

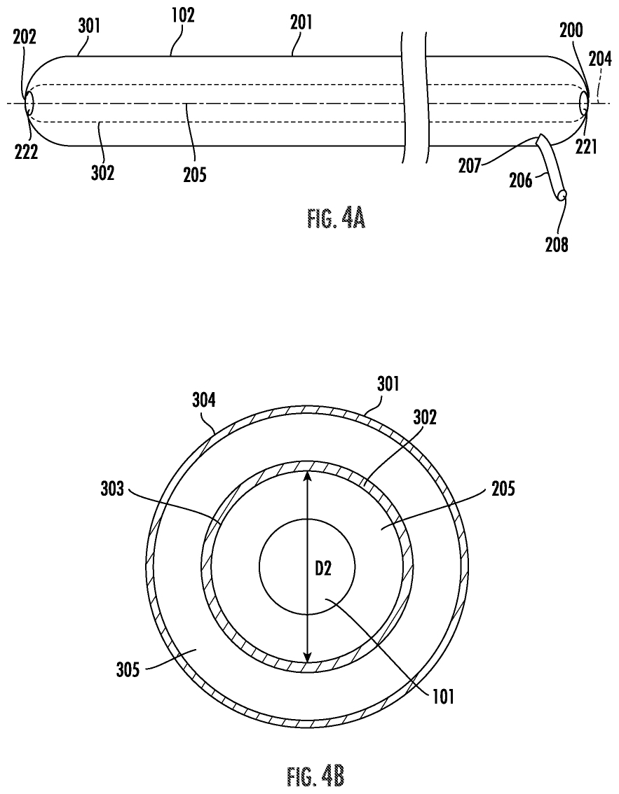

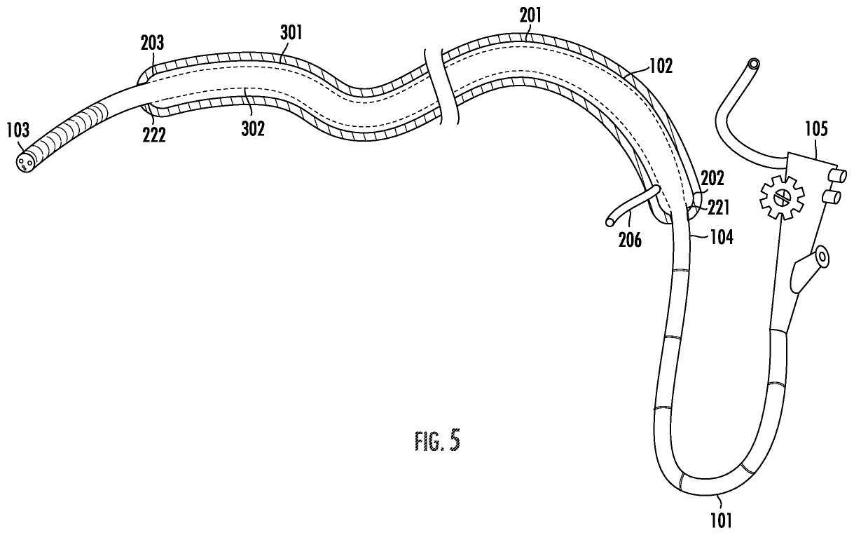

FIGS. 3A and 3B, illustrate respective perspective and cross-sectional views of one embodiment of an instrument accessory device 102 in a collapsed configuration. The instrument accessory device 102 includes the expandable member 201 having a proximal end 200, a distal end 202, and a longitudinal axis 204. An instrument lumen 205 extends along the longitudinal axis 204 of the expandable member 201, extending between the proximal 200 and distal 202 ends and defined by an inner surface 302 of the expandable member 201. The instrument lumen 205 comprises an inlet 221 at the proximal end 200 and an outlet 222 at the distal end 202. The instrument lumen 205 is configured to receive an instrument, e.g., at least a portion of the flexible tubing of the colonoscope of FIG. 1A, extendable through the instrument lumen 205. For example, the instrument lumen 205, in a collapsed configuration, may have a diameter that corresponds generally to a diameter of the colonoscope 101. An outer surface 301 defining an outer diameter of the expandable member 201 extends between the proximal 202 and distal 203 ends along a longitudinal axis 204. The longitudinal axis of the instrument lumen 205 and the longitudinal axis of the expandable member may be generally parallel to each other, as shown. The instrument lumen 204 and the expandable member 201 may each have a center axis along their respective longitudinal axis that may be aligned or offset relative to each other. The outer diameter 304 is disposed about the inner diameter 303, extending between the distal end 203 and the proximal end 202 of the expandable member 201. The accessory devices of FIGS. 3A and 3B, include fluid inlet 206 having a proximal end 208, a distal end 207, and an inflation lumen extending therethrough. In some embodiments, the distal end 207 of the fluid inlet 206 may extend through the outer surface 301 of the expandable member 201. In some embodiments, inner surface 302 of the expandable member 201 is collapsed when the expandable member 201 is collapsed. The inner surface 302 may be a material, e.g., elastic, semi-elastic, weave, mesh, or the like, which can change in dimension. The inner surface 302 may lie flush along the colonoscope when collapsed, or, in some embodiments, may form folds along the colonoscope when collapsed. The inner surface 302 is joined to the outer surface 301 at the distal 203 and proximal 202 ends of the expandable member 201. The surfaces may be joined by an adhesive, a tape, flowed material, welding (e.g., laser, ultrasonic, hot jaw thermal, etc.), melt/re-melt flow processes, or the like. The joined surfaces create a substantially tight fluid seal between the inner surface 302 and the outer surface 301 of the expandable member 201 such that an inflation fluid may be supplied through the inflation lumen of the fluid inlet 206 to transition the expandable member between the collapsed configuration of FIGS. 3A and 3B and an expanded configuration of FIGS. 4A and 4B. The inner surface 302 and outer surface 301 define a chamber 305 which can be inflated by the fluid inserted into the fluid inlet 206.

FIGS. 4A and 4B illustrate the embodiment of 3A and 3B in an expanded configuration. The expandable member 201 in the expanded configuration is illustrated in an elongated ellipsoidal shape, but the expandable member 201 may be other shapes such as, e.g., a sphere, a torus, a combination thereof, or the like. When the expandable member 201 is collapsed, seen in the cross-sectional view of FIG. 3B, the inner diameter 303 of the inner surface 302 is a smaller distance D1 than when the expandable member 201 is expanded and the inner diameter 303 of the inner surface 302 is a larger distance D2, seen in the cross-sectional view of 4B. When in the expanded configuration, an inflation fluid is input through the fluid inlet 206. A colonoscope extending through the instrument lumen 205 of the expandable member 201 may be used to examine a body lumen, as the inner surface 302 remains in frictional contact with the colonoscope when the expandable member 201 is collapsed. With the expandable member 201 in the expanded configuration in a body lumen, the colonoscope may be translated within the instrument lumen 205, as the inner diameter 303 is larger than when the expandable member 201 is collapsed, removing the frictional contact between the colonoscope and the inner surface 302 of the expandable member 201. The frictional contact between the colonoscope and the inner surface 302 allow the instrument accessory device 102 to move through a procedure in concert with the colonoscope until the expandable member 201 is expanded.

The instrument accessory device 102 is illustrated in FIGS. 4A and 4B such that it is configured to receive a colonoscope 101 through the proximal end 202 toward the distal end 203. The expandable member 201 of the instrument accessory device 102 is shown expanded and thus the instrument lumen 205 is configured to slidingly receive the colonoscope 101.

FIG. 5 illustrates the instrument accessory device 102 in a collapsed configuration as part of a colonoscope system 110 in one embodiment. A handle 105 is disposed at the proximal end 104 of the colonoscope 101. The instrument accessory device 102 extends toward the proximal end 104 of the colonoscope 101, such that a portion of the instrument accessory device 102 remains outside of the patient at all times. When in the collapsed configuration, the expandable member 201 is frictionally disposed about the colonoscope 101. The distal end 103 of the colonoscope 101 extends further than the distal end 203 of the instrument accessory device 102, past the outlet 222. The colonoscope 101 extends through the instrument lumen 205, in contact with the inner surface 302. Both the inner surface 302 and outer surface 301 are collapsed. Extending proximal to the outlet 221 of the instrument accessory device 102 is the proximal end 104 of the colonoscope 101, leading to the handle 105. At the proximal end 202 of the instrument accessory device 102 is the fluid inlet 206. In the expanded configuration, the larger diameter of the inner surface 302 allows for a space between the colonoscope 101 and the inner surface 302 in the instrument lumen 205. The outer surface 301 is expandable outward to dilate the body lumen.

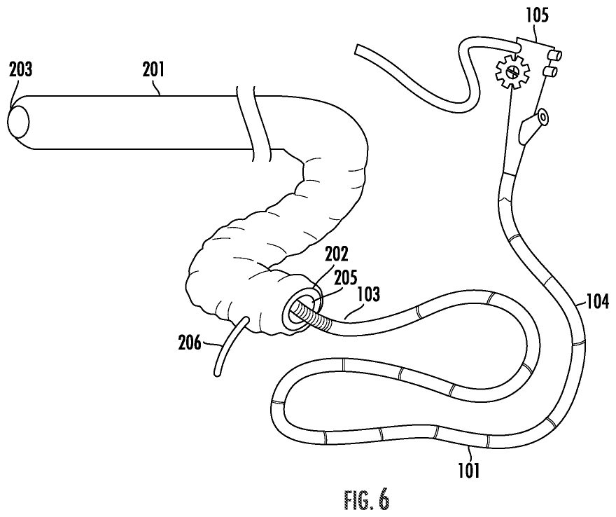

With reference to FIG. 6, a view of an instrument accessory device during the introduction of an instrument prior to insertion into a lumen of the body is illustrated according to an embodiment of the present disclosure. An instrument accessory device 102 with an expanded expandable member 201 can accept the distal end 103 of an instrument through the proximal end 202 of the instrument accessory device 102, with the proximal end 104 of the colonoscope 101 and the handle 105 remaining outside of the instrument accessory device 102. In the expanded state, an instrument can be freely translated within the instrument lumen 205 due to an increased inner diameter 303 of the inner surface 302. This increased inner diameter allows for movement of the instrument within the instrument lumen 205. Once extended through the proximal end 202 of the instrument accessory device 102 and through the instrument lumen 205, the expandable member 201 can be collapsed, and the system resembles that of FIG. 5A.

In various embodiments, an expandable member may have an expanded configuration and a collapsed configuration. An expandable member may comprise an outer surface and an inner surface. An outer surface may comprise a variety of compliant, semi-compliant, or non-compliant materials. These materials may comprise silicone, latex, polyurethane, rubber, isobutylene or the like. The thickness of a wall of the outer surface may vary with the material and may relate to the outer diameter of the outer surface in the collapsed and the expanded configuration. An inner surface may comprise a variety of compliant, semi-compliant, or non-compliant materials. These materials may comprise silicone, latex, polyurethane, rubber, isobutylene, thermoplastic elastomers such as PEBA or Nylon, or the like. The thickness of a wall of the inner surface may vary with the material and may relate to the inner diameter of the inner surface in the collapsed and the expanded configuration. The inner surface may be folded upon itself when in a collapsed configuration. The inner surface and outer surface may not expand to the same degree. An expandable member may be expanded and collapsed or otherwise stretched once or a plurality of times to increase its elasticity prior to use within a patient, which may improve a symmetrical inflation of the expandable member and may improve the centering mechanics of the expandable member. An expandable member may be expanded via a supply of an inflation fluid through one or more fluid inlets. The same fluid inlet may be used to expand and un-expand the expandable member. Alternatively, a supply fluid inlet and a return fluid inlet may be intermittently or continuously used to circulate inflation fluid through an expandable member. A continuous flow of inflation fluid through a supply fluid inlet and a return fluid inlet may substantially maintain a desired pressure of the inflation fluid within the expandable member, or the inflation fluid may be heated and circulated to maintain a desired temperature with the expandable member.

In various embodiments, the inner surface may expand to the same degree as the outer surface. In alternate embodiments, the inner surface may expand to a lesser degree than the outer surface. The inner and outer surfaces may comprise a polymeric material. When expanded, the inner surface may allow an instrument to move slidingly within the instrument lumen and independently of the accessory.

In various embodiments, the instrument accessory device may be disposable. In alternate embodiments, the instrument accessory device may be reusable. In some embodiments, the colonoscope may be a single use device, including the instrument accessory pre-loaded onto the colonoscope prior to use.

In various embodiments, the length of the instrument accessory device is approximately the same as the instrument.

In various embodiments, an inflation fluid supplied through an elongated delivery member may include saline, water, CO₂, dilute contrast media, N₂, O₂, or the like.

In various embodiments, a method of performing a colonoscopy may include expanding an expandable instrument accessory. Once the expandable member of the instrument accessory is expanded, the instrument is advanced through the instrument lumen while the expandable member is in an expanded state. The instrument is advanced until the distal end of the instrument protrudes from the instrument accessory. The expandable member is collapsed such that it is disposed about the instrument and configured to maintain frictional contact with the instrument. The instrument and instrument accessory device are together inserted into a patient. The instrument can be advanced through the patient to the sigmoidal colon and/or the transverse colon, where looping of the instrument may occur. The expandable member can be expanded within the patient, dilating the patient's body lumen, e.g., the colon, the intestines, or the like. The instrument lumen increases in diameter when the expandable member is expanded, allowing the instrument to be adjusted. The expandable member is then contracted, and the colonoscopy exam continued. Expanding the expandable member allows the instrument to move without movement of the instrument accessory. This can occur due to the outer diameter dilating the body lumen, preventing movement of the device. The instrument may be any device used to perform a colonoscopy, e.g., a colonoscope or the like.

In various embodiments, a method of performing a colonoscopy may include expandable an expandable instrument accessory to adjust an instrument. If the instrument becomes looped, the expandable member can be expanded within the patient, dilating the patient's body lumen, e.g., the colon, the intestines, or the like. The instrument lumen increases in diameter when the expandable member is expanded, allowing the instrument to be adjusted and the loop uncoiled. The expandable member is then collapsed, and the colonoscopy exam continued. Expanding the expandable member allows the instrument to move without movement of the instrument accessory. This can occur due to the outer diameter dilating the body lumen, preventing movement of the device.

Variations, modifications, and other implementations of the present disclosure in addition to the various embodiments described herein will occur to those of ordinary skill in the art. Accordingly, the present disclosure is to be defined not by the preceding illustrative description but instead by the following claims:

What is claimed is:

1. A device, comprising:
an expandable member having a proximal end, a distal end, and an instrument lumen extending longitudinally therethrough, the expandable member comprising an inner surface defining the instrument lumen and an outer surface disposed about the inner surface,
wherein the inner surface comprises a material characterized by a first elasticity and the outer surface comprises a material characterized by a second elasticity, the second elasticity being more elastic than the first elasticity.

2. The device of claim 1, wherein the instrument lumen is configured to receive the instrument when the expandable member is in an expanded configuration.

3. The device of claim 1, wherein the instrument lumen is configured to maintain frictional contact with the instrument when the expandable member is in a collapsed configuration.

4. The device of claim 1, wherein, when, the expandable member comprises the collapsed configuration, an expandable member diameter, an instrument lumen diameter, or both, correspond to a diameter of the instrument, and, when the expandable member comprises the expanded configuration, the expandable member diameter, the instrument lumen diameter, or both, are larger the diameter of the instrument.

5. The device of claim 1, wherein the inner surface and outer surface are joined to form a closed chamber.

6. A system, comprising:
a colonoscope; and
a colonoscope accessory disposable about the colonoscope, comprising:
an expandable member having a proximal end, a distal end, and an instrument lumen extending longitudinally therethrough, the expandable member comprising an inner surface defining the instrument lumen and an outer surface disposed about the inner surface;
wherein the inner surface comprises a material characterized by a first elasticity and the outer surface comprises a material characterized by a second elasticity, the second elasticity being more elastic than the first elasticity;
wherein the instrument lumen is configured to slidingly receive at least a portion of the colonoscope when the expandable member is in an expanded condition.

7. The system of claim 6, wherein the expandable member maintains frictional contact with the colonoscope when the expandable member is in a collapsed condition.

8. The system of claim 6, wherein the material of the outer surface is compliant and configured to expand to conform to a body cavity.

9. The system of claim 6, wherein when the expandable member is in an expanded condition, an inner diameter of the instrument lumen allows the colonoscope to move freely within the instrument lumen.

10. The system of claim 6, wherein the expandable member is pneumatically, electrically, or mechanically expandable.

11. A method of performing a procedure within a body lumen, comprising:
advancing an instrument at least partway into the body lumen, the instrument comprising an instrument accessory device having an expandable member disposed about at least a distal portion of the instrument, the expandable member having a proximal end, a distal end, and an instrument lumen extending longitudinally therethrough, the expandable member comprising an inner surface defining the instrument lumen and an outer surface disposed about the inner surface, the expandable member having a collapsed configuration wherein one or both of an expandable member diameter and an instrument lumen diameter corresponds to a diameter of the instrument and an expanded configuration wherein one or both of the expandable member diameter and the instrument lumen diameter are larger than the diameter of the instrument;
expanding the expandable member to the expanded configuration; and
continuing to advance or retract the instrument through the instrument lumen within the body lumen while the expandable member is in the expanded configuration.

12. The method of claim 11, wherein expanding the expandable member within the body lumen expands an inner diameter of the instrument lumen such that the instrument is able to move within the instrument lumen of the expandable member.

13. The method of claim 11, wherein expanding the expandable member allows the instrument to translate within the instrument lumen.

14. The method of claim 11, wherein the step of expanding the expandable member comprises injecting a gas or a liquid into the expandable member.

15. The method of claim 11, further comprising the step of collapsing the expandable member by removing a gas or liquid from the expandable member.

16. The method of claim 11, wherein the outer surface comprises a compliant material that is expandable to conform to the body lumen.

17. The method of claim 11, wherein expansion of an outer diameter of the expandable member inhibits movement of the expandable member within the body lumen.

18. The method of claim 11, wherein the body lumen comprises a colon and the instrument comprises a colonoscope.

19. The method of claim 11, wherein the proximal end of the expandable member remains outside of the body lumen during the procedure.

20. The method of claim 11, wherein expanding the expandable member preferentially expands an outer diameter of the expandable member.
